SEQUENCE LISTING (13 sequences)

SEQ ID NO: 1 - protein, 30 aa, Artificial
  Conus spp
XXXLXPFXYYXLXRYXTRFLHXXPXYYXXX

SEQ ID NO: 2 - protein, 30 aa, Conus planorbis
ARFLHPFQYYTLYRYLTRFLHRYPIYYIRY

SEQ ID NO: 3 - protein, 30 aa, Conus ferrugineus
GTYLYPFSYYRLWRYFTRFLHKQPYYYVHI

SEQ ID NO: 4 - protein, 26 aa, Conus planorbis
SLKNEDGSIPYFMPHILKYLRLYYYH

SEQ ID NO: 5 - protein, 23 aa, Conus betulinus
VLKRSWIYHVRPHYSANTLWSLV

SEQ ID NO: 6 - protein, 36 aa, Homo sapiens
YPSKPDNPGEDAPAEDMARYYSALRHYINLITRQRY

SEQ ID NO: 7 - protein, 9 aa, Conus planorbis
NNPREDICT

SEQ ID NO: 8 - DNA, 63 bp, Conus planorbis
  CDS (1)..(63)
atgatgtcgaaactgggagtcgtgctgttcgtctttctgcttctgcttcctctggcggctcct

SEQ ID NO: 9 - protein, 21 aa, Conus planorbis
MMSKLGVVLFVFLLLLPLAAP

SEQ ID NO: 10 - DNA, 63 bp, Conus betulinus
  CDS (1)..(63)
atgatgtcgaaactgggagtcgtattgttcatctttctggttctgtttcccatggcaactctt

SEQ ID NO: 11 - protein, 21 aa, Conus betulinus
MMSKLGVVLFIFLVLFPMATL

SEQ ID NO: 12 - protein, 19 aa, Conus planorbis
QPVGDQPADQPADRNAEAR

SEQ ID NO: 13 - DNA, 63 bp, Artificial
  Conus consensus sequence
atgatgtcgaaactgggagtcgtrytgttcrtctttctgnttctgyttccyntggcrrctcyt

(19) United States
(12) Patent Application Publication
(10) Pub. No.: US 2010/0093620 A1
(43) Pub. Date: Apr. 15, 2010

(54) TYROSINE-RICH CONOPEPTIDES

(75) Inventors: Julita S. IMPERIAL, Midvale, UT (US); Baldomero M. OLIVERA, Salt Lake City, UT (US); Heinrich TERLAU, Eckernfoerde (DE); Paul F. ALEWOOD, Brisbane (AU); David J. CRAIK, Brisbane (AU)

Correspondence Address: ROTHWELL, FIGG, ERNST & MANBECK, P.C., 1425 K STREET, N.W., SUITE 800, WASHINGTON, DC, 20005, US

(73) Assignee: UNIVERSITY OF UTAH RESEARCH FOUNDATION, Salt Lake City, UT (US)

(21) Appl. No.: 12/433,037

(22) Filed: Apr. 30, 2009

Related U.S. Application Data
(60) Provisional application No. 61/049,106, filed on Apr. 30, 2008.

(51) Int. Cl. A61K 38/16; C07K 14/00; G01N 33/53; A61P 3/10; A61P 9/12

(52) U.S. Cl. 514/12; 530/324; 436/86

(57) ABSTRACT

The invention relates to relatively short peptides (termed Conopeptide-Y family peptides or CPY family peptides or CPY peptides herein), about 30 residues in length, which are naturally available in minute amounts in the venom of the cone snails or analogous to the naturally available peptides, and which are rich in the amino acid tyrosine.

### CROSS-REFERENCE TO RELATED APPLICATION

The present application is related to and claims priority under 35 U.S.C. §119(e) to U.S. provisional patent application Ser. No. 61/049,106 filed on 30 Apr. 2008. This application is incorporated herein by reference.

### REFERENCE TO GOVERNMENT SUPPORT

This invention was made with Government support under Grant No. PO1 GM48677 awarded by the National Institute of General Medical Sciences, National Institutes of Health, Bethesda, Md. The United States Government has certain rights in the invention.

### BACKGROUND OF THE INVENTION

The invention relates to relatively short peptides (termed Conopeptide-Y family peptides or CPY family peptides or CPY peptides herein), about 30 residues in length, which are naturally available in minute amounts in the venom of the cone snails or analogous to the naturally available peptides, and which are rich in the amino acid tyrosine.

The publications and other materials used herein to illuminate the background of the invention, and in particular, cases to provide additional details respecting the practice, are incorporated by reference, and for convenience are referenced in the following text by number and are listed by number in the appended bibliography.

The venom of marine gastropods in the genus Conus has yielded numerous structurally and functionally diverse peptidic components (1). The increasing variety of bioactive peptides identified in cone snail venoms has provided insight into the seemingly endless variety of directions taken by Conus species in evolving neuroactive molecules to suit their diverse biological purposes.

The bioactive peptides in Conus (“conopeptides”) are classified into two broad groups: the non-disulfide-rich and the disulfide-rich (1); the latter are conventionally called conotoxins. The non-disulfide-rich class includes conopeptides with no cysteines (contulakins (2), conantokins (3), and conorfamides (4)), and conopeptides with two cysteines forming a single disulfide bond (conopressins (5) and contryphans (6)). The conopeptides that comprise the disulfide-rich class have two or more disulfide bonds (1); among the major classes of molecular targets identified for these structurally diverse conopeptides are members of the voltage-gated and ligand-gated ion channel superfamilies.

However, the structure and function of only a small minority of these peptides have been determined to date. For peptides where function has been determined, three classes of targets have been elucidated: voltage-gated ion channels; ligand-gated ion channels, and G-protein-linked receptors.

Conus peptides which target voltage-gated ion channels include those that delay the inactivation of sodium channels, as well as blockers specific for sodium channels, calcium channels and potassium channels. Peptides that target ligand-gated ion channels include antagonists of NMDA and serotonin receptors, as well as competitive and noncompetitive nicotinic receptor antagonists. Peptides which act on G-protein receptors include neurotensin and vasopressin receptor agonists. The unprecedented pharmaceutical selectivity of conotoxins is at least in part defined by specific disulfide bond frameworks combined with hypervariable amino acids within disulfide loops (for a review see McIntosh et al. (28)).

In view of a large number of biologically active substances in Conus species it is desirable to further characterize them and to identify peptides capable of treating disorders involving voltage-gated ion channels, ligand-gated ion channels and/or receptors. Surprisingly, and in accordance with this invention, Applicants have discovered novel conopeptides that can be useful for the treatment of disorders involving potassium channels and could address a long felt need for a safe and effective treatment.

### SUMMARY OF THE INVENTION

The invention relates to relatively short peptides (termed Conopeptide-Y family peptides or CPY family peptides or CPY peptides herein), about 30 residues in length, which are naturally available in minute amounts in the venom of the cone snails or analogous to the naturally available peptides. Peptides in the CPY family have a high content of tyrosine accompanied by the preponderance of the amino acids phenylalanine, proline, the basic amino acids lysine, arginine and/or histidine, and the strongly hydrophobic amino acids leucine, isoleucine and/or valine. These peptides are encoded by genes, which share a conserved signal sequence.

More specifically, the present invention is directed to CPY peptides having the general formula I:

(SEQ ID NO: 1)

Xaa1-Xaa2-Xaa3-Leu-Xaa5-Pro-Phe-Xaa8-Tyr-Tyr-
Xaa11-Leu-Xaa13-Arg-Tyr-Xaa16-Thr-Arg-Phe-Leu-His-
Xaa22-Xaa23-Pro-Xaa25-Tyr-Tyr-Xaa28-Xaa29-Xaa30,

wherein Xaa1 is Gly, Ala, an aliphatic amino acids bearing linear or branched saturated hydrocarbon chains such as Leu (D or L), Ile and Val or non-natural derivatives of the aliphatic amino acid; Xaa2 is Thr, g-Thr (where g is glycosylation), Ser, g-Ser, Arg, Lys, ornithine, homo-Lys, homoarginine, nor-Lys, N-methyl-Lys, N,N′-dimethyl-Lys, N,N′,N″-trimethyl-Lys or any synthetic basic amino acid; Xaa3 is Phe, Tyr, meta-Tyr, ortho-Tyr, nor-Tyr, mono-halo-Tyr, di-halo-Tyr, O-sulpho-Tyr, O-phospho-Tyr, nitro-Tyr; Xaa5 is His, Tyr, meta-Tyr, ortho-Tyr, nor-Tyr, mono-halo-Tyr, di-halo-Tyr, O-sulpho-Tyr, O-phospho-Tyr, nitro-Tyr; Xaa8 is Ser, g-Ser, Thr, g-Thr, Gln, Asn; Xaa11 is Thr, g-Thr, Ser, g-Ser, Arg, Lys, ornithine, homo-Lys, homoarginine, nor-Lys, N-methyl-Lys, N,N′-dimethyl-Lys, N,N′,N″-trimethyl-Lys or any synthetic basic amino acid; Xaa13 is Trp (D or L), halo-Trp (D or L), Tyr, meta-Tyr, ortho-Tyr, nor-Tyr, mono-halo-Tyr, di-halo-Tyr, O-sulpho-Tyr, O-phospho-Tyr, nitro-Tyr; Xaa16 is Phe, an aliphatic amino acids bearing linear or branched saturated hydrocarbon chains such as Leu (D or L), Ile and Val or non-natural derivatives of the aliphatic amino acid; Xaa22 is Lys, Arg, ornithine, homo-Lys, homoarginine, nor-Lys, N-methyl-Lys, N,N′-dimethyl-Lys, N,N′,N″-trimethyl-Lys or any synthetic basic amino acid; Xaa23 is Gln, Asn, Tyr, meta-Tyr, ortho-Tyr, nor-Tyr, mono-halo-Tyr, di-halo-Tyr, O-sulpho-Tyr, O-phospho-Tyr, nitro-Tyr; Xaa25 is an aliphatic amino acids bearing linear or branched saturated hydrocarbon chains such as Leu (D or L), Ile and Val or non-natural derivatives of the aliphatic amino acid, Tyr, meta-Tyr, ortho-Tyr, nor-Tyr, mono-halo-Tyr, di-halo-Tyr, O-sulpho-Tyr, O-phospho-Tyr, nitro-Tyr; Xaa28 is an aliphatic amino acids bearing linear or branched saturated hydrocarbon chains such as Leu (D or L), Ile and Val or non-natural derivatives of the aliphatic amino acid; Xaa29 is His, Arg, Lys, ornithine, homo-Lys, homoarginine, nor-Lys, N-methyl-Lys, N,N′-dimethyl-Lys, N,N′,N″-trimethyl-Lys or any synthetic basic amino acid; and Xaa30 is an aliphatic amino acids bearing linear or branched saturated hydrocarbon chains such as Leu (D or L), Ile and Val or non-natural derivatives of the aliphatic amino acid, Tyr, meta-Tyr, ortho-Tyr, nor-Tyr, mono-halo-Tyr, di-halo-Tyr, O-sulpho-Tyr, O-phospho-Tyr, nitro-Tyr.

The Tyr residues may be substituted with the 3-hydroxyl or 2-hydroxyl isomers and corresponding O-sulpho- and O-phospho-derivatives. The non-natural derivatives of the aliphatic amino acids include those synthetic derivatives bearing non-natural aliphatic branched or linear side chains CₙH₂ₙ₊₂ up to and including n=8. The halogen is iodo, chloro, fluoro or bromo; preferably iodo for halogen substituted-Tyr and bromo for halogen-substituted Tip. In addition, the halogen can be radiolabeled, e.g., ¹²⁵I-Tyr.

The present invention is also directed to novel specific CPY peptides having the mature toxin sequences set forth in Table 1.

TABLE 1
CPY Peptides

| | |
|---|---|
| CPY-P11 | ARFLHPFQYYTLYRYLTRFLHRYPIYYIRY |
| | (SEQ ID NO: 2) |
| CPY-Fe1 | GTYLYPFSYYRLWRYFTRFLHKQPYYYVHI |
| | (SEQ ID NO: 3) |
| CPY-p12 | SLKNEDGSIPYFMPHILKYLRLYYYH |
| | (SEQ ID NO: 4) |
| CPY-be1 | VLKRSWIYHVRPHYSANTLWSLV |
| | (SEQ ID NO: 5) |

In addition, the present invention is directed to the above CPY peptides of the general formula I or the CPY peptides set forth in Table 1 in which the Pro residues may be substituted with hydroxyl-Pro; the Arg residues may be substituted by Lys, ornithine, homoargine, nor-Lys, N-methyl-Lys, N,N-dimethyl-Lys, N,N,N-trimethyl-Lys or any synthetic basic amino acid; the Lys residues may be substituted by Arg, ornithine, homoargine, nor-Lys, or any synthetic basic amino acid; the Tyr residues may be substituted with any synthetic hydroxy containing amino acid; the Ser residues may be substituted with Thr or any synthetic hydroxylated amino acid; the Thr residues may be substituted with Ser or any synthetic hydroxylated amino acid; the Phe and Trp residues may be substituted with any synthetic aromatic amino acid; and the Asn, Ser, Thr or Hyp residues may be glycosylated. The Tyr residues may also be substituted with the 3-hydroxyl or 2-hydroxyl isomers (meta-Tyr or ortho-Tyr, respectively) and corresponding O-sulpho- and O-phospho-derivatives. The aliphatic amino acids may be substituted with one another or with Ala or with synthetic derivatives bearing non-natural aliphatic branched or linear side chains CₙH₂ₙ₊₂ up to and including n=8. The Leu residues may be substituted with Leu (D). The Tip residues may be substituted with Tip (D).

Examples of synthetic aromatic amino acid include, but are not limited to, such as nitro-Phe, 4-substituted-Phe wherein the substituent is C₁-C₃ alkyl, carboxyl, hyrdroxymethyl, sulphomethyl, halo, phenyl, —CHO, —CN, —SO₃H and —NHAc. Examples of synthetic hydroxy containing amino acid, include, but are not limited to, such as 4-hydroxymethyl-Phe, 4-hydroxyphenyl-Gly, 2,6-dimethyl-Tyr and 5-amino-Tyr. Examples of synthetic basic amino acids include, but are not limited to, N-1-(2-pyrazolinyl)-Arg, 2-(4-piperinyl)-Gly, 2-(4-piperinyl)-Ala, 2-[3-(2S)pyrrolininyl)-Gly and 2-[3-(2S)pyrrolininyl)-Ala. These and other synthetic basic amino acids, synthetic hydroxy containing amino acids or synthetic aromatic amino acids are described in Building Block Index, Version 3.0 (1999 Catalog, pages 4-47 for hydroxy containing amino acids and aromatic amino acids and pages 66-87 for basic amino acids; see also the website “amino-acids dot com”), incorporated herein by reference, by and available from RSP Amino Acid Analogues, Inc., Worcester, Mass.

Optionally, in the peptides of general formula I and the specific peptides described above, the Asn residues may be modified to contain an N-glycan and the Ser, Thr and Hyp residues may be modified to contain an O-glycan (e.g., g-N, g-S, g-T and g-Hyp). In accordance with the present invention, a glycan shall mean any N-, S- or O-linked mono-, di-, tri-, poly- or oligosaccharide that can be attached to any hydroxy, amino or thiol group of natural or modified amino acids by synthetic or enzymatic methodologies known in the art. The monosaccharides making up the glycan can include D-allose, D-altrose, D-glucose, D-mannose, D-gulose, D-idose, D-galactose, D-talose, D-galactosamine, D-glucosamine, D-N-acetyl-glucosamine (GlcNAc), D-N-acetyl-galactosamine (GalNAc), D-fucose or D-arabinose. These saccharides may be structurally modified, e.g., with one or more O-sulfate, O-phosphate, O-acetyl or acidic groups, such as sialic acid, including combinations thereof. The gylcan may also include similar polyhydroxy groups, such as D-penicillamine 2,5 and halogenated derivatives thereof or polypropylene glycol derivatives. The glycosidic linkage is β and 1-4 or 1-3, preferably 1-3. The linkage between the glycan and the amino acid may be α or β, preferably α and is 1-.

Core O-glycans have been described by Van de Steen et al. (29), incorporated herein by reference. Mucin type O-linked oligosaccharides are attached to Ser or Thr (or other hydroxylated residues of the present peptides) by a GalNAc residue. The monosaccharide building blocks and the linkage attached to this first GalNAc residue define the “core glycans,” of which eight have been identified. The type of glycosidic linkage (orientation and connectivities) are defined for each core glycan. Suitable glycans and glycan analogs are described further in U.S. Pat. No. 6,369,193 and in PCT Published Application No. WO 00/23092, each incorporated herein by reference. A preferred glycan is Gal(β1→3)GalNAc(α1→).

The present invention is also directed to the identification of the nucleic acid sequences encoding these peptides and their propeptides and the identication of nucleic acid sequences of additional related CPY peptides. Thus, the present invention is directed to nucleic acids coding for the CPY peptide precursors (or CPY propeptides) set forth herein. The present invention is further directed to the CPY propeptides set forth herein.

The present invention is further directed to a method of treating disorders associated with voltage gated ion channel or receptor disorders in a subject comprising administering to the subject an effective amount of the composition comprising a therapeutically effective amount of a CPY peptide described herein or a pharmaceutically acceptable salt or solvate thereof. The present invention is also directed to a composition comprising a therapeutically effective amount of a CPY peptide described herein or a pharmaceutically acceptable salt or solvate thereof and a pharmaceutically acceptable carrier.

Another embodiment of the invention contemplates a method of identifying compounds that mimic the therapeutic activity of the instant peptide, comprising the steps of: (a) conducting a biological assay on a test compound to determine the therapeutic activity; and (b) comparing the results obtained from the biological assay of the test compound to the results obtained from the biological assay of the peptide. In relation to radioligand probes of CPY peptides for screening of small molecules, acting at unique allosteric sites, synthesis of such screening tools is not restricted to radioiodinated tyrosine derivatives. Incorporation of standard commercially available tritiated amino acid residues can also be utilized.

### BRIEF DESCRIPTION OF THE FIGURES

FIGS. 1A-1C show the isolation of CPY-Pl1 and CPY-Fe1 from an extract of C. planorbis venom. FIG. 1A: Preparative HPLC chromatogram of the extract showing the absorbance profile at 220 nm. The preparation of extract and the HPLC parameters used were as described in Experimental Procedures. The peak indicated by the arrow was potent on the marine worm N. vixens as described in Results. FIG. 1B: Analytical HPLC chromatogram showing the absorbance profile of subfractionation of the active peak in A. The arrow marks the active subfraction. FIG. 1C: Analytical HPLC chromatogram of the alkylation reaction mixture of the active subfraction from B. Peptide sequencing of the peaks denoted by arrows yielded the sequences for CPY-Pl1 (peak on the left) and CPY-Fe1 (peak on the right).

FIG. 2 shows an alignment of CPY peptides with Neuropeptide Y (NPY) (SEQ ID NO:6).

FIGS. 3A-3B show coleution (right) of CPY-Fe1 (FIG. 3A) and CPY-Pl1 (FIG. 3B) that were isolated from the venom (left) and the corresponding synthetic peptides (middle). HPLC was performed in an analytical C18 column using a gradient of 0.18% acetonitrile/min-0.1% TFA in a Waters Millenium HPLC system with auto sampler and data were transcribed into Prism.

FIG. 4 shows chemical shift analysis of CPY-Pl1 (SEQ ID NO:2). The secondary shifts were determined by subtracting the random coil shifts (27) from the experimental αH shifts of CPY-Pl1. Values in aqueous solution are shown with white bars and those in the presence of 20% TFE with black bars. The dashed line at −0.1 ppm indicates the threshold for significant deviations from random coil. The helical region in the presence of TFE from residues 12 to 18 is highlighted schematically. The predicted helical region (NNPREDICT) (SEQ ID NO:7) is shown with a grey bar and overlaps significantly with the helical region deduced from the chemical shifts.

FIG. 5 shows activity of CPY-Fe1 and CPY Pl1 on the Kv1.6 channel. Each data point is an average of responses obtained from at least three oocytes as described in Methods and the dose-response curves were generated using Prism. The IC₅₀ for CPY-Fe1 is 8.8 μM and the IC₅₀ for CPY-Pl1 is 170 nM.

FIG. 6 shows CPY-Pl1 and CPY-Fe1 precursors. Complete homology between the two clones occurs in the signal sequence and propeptide regions; ˜91% homology is observed in the 3′UTRs. The residues shared by the mature peptides are in bold letters.

### DETAILED DESCRIPTION OF THE INVENTION

The invention relates to relatively short peptides (termed Conopeptide-Y family peptides or CPY family peptides or CPY peptides herein), about 30 residues in length, which are naturally available in minute amounts in the venom of the cone snails or analogous to the naturally available peptides. Peptides in the CPY family have a high content of tyrosine accompanied by the preponderance of the amino acids phenylalanine, proline, the basic amino acids lysine, arginine and/or histidine, and the strongly hydrophobic amino acids leucine, isoleucine and/or valine. These peptides are encoded by genes, which share a conserved signal sequence.

This application presents the discovery and characterization of a novel class of peptides, which we designate the Conopeptide-Y (CPY) family, from the marine snails Conus planorbis and Conus ferrugineus. Both of these species belong to a distinct clade of Conus (7,8). The first two peptides (9) belonging to the CPY family have a high frequency of tyrosine in addition to basic and hydrophobic residues. The peptides were shown to be biologically active when injected into mouse and the nematode C. elegans. Activity on Kv1 channel subfamily was demonstrated, and the subtype selectively within the subfamily was defined.

The CPY peptides of the present invention include those described above. As noted, the peptides of this family have a high content of certain amino acids. Table 2 shows the frequencies of the more predominant amino acids in the peptide described herein.

TABLE 2
Frequencies of More Predominant Amino Acids in CPY Peptides

| | |
|---|---|
| Peptide | % amino acid residues in the peptide |

| (SEQ ID NO:) | Y | I + V + L + F | H + K + R | P |
|---|---|---|---|---|
| 2 | 27 | 30 | 23 | 7 |
| 3 | 27 | 27 | 20 | 7 |
| 4 | 19 | 27 | 19 | 8 |
| 5 | 9 | 30 | 22 | 4 |

The present invention is also directed to cDNA clones encoding the precursor of the biologically-active mature peptides and to the precursor peptides.

The present invention is further directed to a consensus sequence encoding the signal peptide of the CPY peptides, as well as the consensus signal peptide. This consensus DNA sequence can be used to clone other members of the CPY family using conventional techniques well known to the skilled artisan, as well as the techniques described herein. The DNA sequence for three of the CPY signal peptides, as well as the consensus sequence is set forth in Table 3. The signal peptide sequences are set forth in SEQ ID NOs:9 and 11, which are the translation products of SEQ ID NO:8 and 10, respectively.

TABLE 3
Alignments of DNA Sequence Encoding Signal Peptide and Consensus Sequence

| | | |
|---|---|---|
| CPY-P11 | ATGATGTCGAAACTGGGAGTCGTGCTGTTCGTCTTTCTGCTTCTGCTTCC | (SEQ ID NO: 8) |
| CPY-Fe1 | ATGATGTCGAAACTGGGAGTCGTGCTGTTCGTCTTTCTGCTTCTGCTTCC | (SEQ ID NO: 8) |
| CPY-be1 | ATGATGTCGAAACTGGGAGTCGTATTGTTCATCTTTCTGGTTCTGTTTCC | (SEQ ID NO: 10) |
| Consensus | ATGATGTCGAAACTGGGAGTCGTRYTGTTCRTCTTTCTGNTTCTGYTTCC | (SEQ ID NO: 13) |
| CPY-P11 | TCTGGCGGCTCCT | |
| CPY-Fe1 | TCTGGCGGCTCCT | |
| CPY-be1 | CATGGCAACTCTT | |
| Consensus | YNTGGCRRCTCYT | |

The present invention, in another aspect, relates to a 1 composition comprising an effective amount of a CPY peptide, a mutein thereof, an analog thereof, an active fragment thereof or pharmaceutically acceptable salts or solvates. Such a composition has the capability of acting at voltage-gated ion channels, ligand-gated ion channels and/or receptors, and are thus useful for treating a disorder or disease of a living animal body, including a human, which disorder or disease is responsive to the partial or complete blockade of such channels or receptors comprising the step of administering to such a living animal body, including a human, in need thereof a therapeutically effective amount of a pharmaceutical composition of the present invention. The CPY peptides are active at the voltage-gated potassium channel.

For example, attention has been focused on the potassium channel, particularly its involvement in normal cellular homeostasis and its possible association with and derangements relating to a variety of disease states and immune responses. Considerable research has been expended and is currently underway in order not only to devise a treatment or prophylaxis against such devastating diseases, but also to study the underlying etiology(ies) such that a better understanding can be gained as to common denominators, if any, that would more directly focus a plan of attack for conquering them. Diseases having a particular association with such channels include autoimmune diseases and other proliferative disorders such as cancers. Autoimmune diseases include rheumatoid arthritis, type-1 diabetes mellitus (insulin dependent), multiple sclerosis, myasthenia gravis, systematic lupus erythematosus, Sjogren's syndrome, mixed connective tissue disease, experimental allergic encephalomyelitis (EAE), to name a few.

Potassium channels comprise a large and diverse group of proteins that, through maintenance of the cellular membrane potential, are fundamental in normal biological function. These channels are vital in controlling the resting membrane potential in excitable cells and can be broadly sub-divided into three classes: voltage-gated K⁺ channels, Ca²⁺ activated K⁺ channels and ATP-sensitive K⁺ channels. Many disorders are associated with abnormal flow of potassium ions through these channels. These disorders include multiple sclerosis, other demyelinating diseases (such as acute dissenmiated encephalomyelitis, optic neuromyelitis, adrenoleukodystrophy, acute transverse myelitis, progressive multifocal leukoencephalopathy), sub-acute sclerosing panencephalomyelitis (SSPE), metachromatic leukodystrophy, Pelizaeus-Merzbacher disease, spinal cord injury, botulinum toxin poisoning, Huntington's chorea, compression and entrapment neuropathies (such as carpal tunnel syndrome, ulnar nerve palsy), cardiovascular disorders (such as cardiac arrhythmias, congestive heart failure), reactive gliosis, hyperglycemia, immunosuppression, cocaine addiction, cancer, cognitive dysfunction, disorders resulting from defects in neurotransmitter release (such as Eaton-Lambert syndrome), and reversal of the actions of curare and other neuromuscular blocking drugs. Other uses of compounds that are active at K⁺ channels are described in U.S. Published Patent Application No. 2002/0102607 A1, U.S. Published Patent Application No. 2004/0092447 A1, U.S. Published Patent Application No. 2005/0137132 A1 and U.S. Published Patent Application No. 2006/0014673 A1, each incorporated herein by reference.

The invention is further directed to the use of these peptides for screening drugs for activity at the receptor of these conopeptides and to isolate and assay receptors.

The conopeptides of the present invention are identified by isolation from Conus venom. Alternatively, the conopeptides of the present invention are identified using recombinant DNA techniques by screening cDNA libraries of various Conus species using conventional techniques such as the use of reverse-transcriptase polymerase chain reaction (RT-PCR) or the use of degenerate probes. Primers for RT-PCR are based on conserved sequences in the signal sequence and 3′ untranslated region of the CPY peptide genes. Clones which hybridize to these probes are analyzed to identify those which meet minimal size requirements, i.e., clones having approximately 300 nucleotides (for a precursor peptide), as determined using PCR primers which flank the cDNA cloning sites for the specific cDNA library being examined. These minimal-sized clones are then sequenced. The sequences are then examined for the presence of a peptide having the characteristics noted above for conopeptides. The biological activity of the peptides identified by this method is tested as described herein, in U.S. Pat. No. 5,635,347 or conventionally in the art.

These peptides are sufficiently small to be chemically synthesized by techniques well known in the art. The peptides are synthesized by a suitable method, such as by exclusively solid-phase techniques (Merrifield solid-phase synthesis), by partial solid-phase techniques, by fragment condensation or by classical solution couplings. Suitable techniques are exemplified by the disclosures of U.S. Pat. Nos. 4,105,603; 3,972,859; 3,842,067; 3,862,925; 4,447,356; 5,514,774; and 5,591,821, each incorporated herein.

Various ones of these CPY peptides can also be obtained by isolation and purification from specific Conus species using the techniques described in U.S. Pat. Nos. 4,447,356; 5,514,774 and 5,591,821, the disclosures of which are incorporated herein by reference.

The CPY peptides can also be produced by recombinant DNA techniques well known in the art. Such techniques are described by Sambrook et al. (30).

Muteins, analogs or active fragments, of the foregoing CPY peptides are also contemplated here. See, e.g., Hammerland et al. (31). Derivative muteins, analogs or active fragments of the CPY peptides may be synthesized according to known techniques, including conservative amino acid substitutions, such as outlined in U.S. Pat. No. 5,545,723 (see particularly col. 2, line 50 to col. 3, line 8); U.S. Pat. No. 5,534,615 (see particularly col. 19, line 45 to col. 22, line 33); and U.S. Pat. No. 5,364,769 (see particularly col. 4, line 55 to col. 7, line 26), each incorporated herein by reference.

Pharmaceutical compositions containing a compound of the present invention as the active ingredient can be prepared according to conventional pharmaceutical compounding techniques. See, for example, Remington: The Science and Practice of Pharmacy, 21st Ed., Lippincott Williams & Wilkins, Philadelphia, 2005. Typically, an antagonistic amount of active ingredient will be admixed with a pharmaceutically acceptable carrier. The carrier may take a wide variety of forms depending on the form of preparation desired for administration, e.g., intravenous, oral, parenteral or intrathecally. For examples of delivery methods see U.S. Pat. No. 5,844,077, incorporated herein by reference.

“Pharmaceutical composition” means physically discrete coherent portions suitable for medical administration. “Pharmaceutical composition in dosage unit form” means physically discrete coherent units suitable for medical administration, each containing a daily dose or a multiple (up to four times) or a sub-multiple (down to a fortieth) of a daily dose of the active compound in association with a carrier and/or enclosed within an envelope. Whether the composition contains a daily dose, or for example, a half, a third or a quarter of a daily dose, will depend on whether the pharmaceutical composition is to be administered once or, for example, twice, three times or four times a day, respectively.

The term “salt”, as used herein, denotes acidic and/or basic salts, formed with inorganic or organic acids and/or bases, preferably basic salts. While pharmaceutically acceptable salts are preferred, particularly when employing the compounds of the invention as medicaments, other salts find utility, for example, in processing these compounds, or where non-medicament-type uses are contemplated. Salts of these compounds may be prepared by art-recognized techniques.

Examples of such pharmaceutically acceptable salts include, but are not limited to, inorganic and organic addition salts, such as hydrochloride, sulphates, nitrates or phosphates and acetates, trifluoroacetates, propionates, succinates, benzoates, citrates, tartrates, fumarates, maleates, methane-sulfonates, isothionates, theophylline acetates, salicylates, respectively, or the like. Lower alkyl quaternary ammonium salts and the like are suitable, as well.

As used herein, the term “pharmaceutically acceptable” carrier means a non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations.

Wetting agents, emulsifiers and lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Examples of pharmaceutically acceptable antioxidants include, but are not limited to, water soluble antioxidants such as ascorbic acid, cysteine hydrochloride, sodium bisulfite, sodium metabisulfite, sodium sulfite, and the like; oil soluble antioxidants, such as ascorbyl palmitate, butylated hydroxyanisole (BHA), butylated hydroxytoluene (BHT), lecithin, propyl gallate, alpha-tocopherol and the like; and the metal chelating agents such as citric acid, ethylenediamine tetraacetic acid (EDTA), sorbitol, tartaric acid, phosphoric acid and the like.

For oral administration, the compounds can be formulated into solid or liquid preparations such as capsules, pills, tablets, lozenges, melts, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier. See for example, WO 96/11698.

For parenteral administration, the compound may be dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

A variety of administration routes are available. The particular mode selected will depend of course, upon the particular drug selected, the severity of the disease state being treated and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, sublingual, topical, nasal, transdermal or parenteral routes. The term “parenteral” includes subcutaneous, intravenous, epidural, irrigation, intramuscular, release pumps, or infusion. For example, administration of the active agent according to this invention may be achieved using any suitable delivery means, including those described in U.S. Pat. No. 5,844,077, incorporated herein by reference.

Alternatively, targeting therapies may be used to deliver the active agent more specifically to certain types of cell, by the use of targeting systems such as antibodies or cell specific ligands. Targeting may be desirable for a variety of reasons, e.g. if the agent is unacceptably toxic, or if it would otherwise require too high a dosage, or if it would not otherwise be able to enter the target cells.

The active agents, which are peptides, can also be administered in a cell based delivery system in which a DNA sequence encoding an active agent is introduced into cells designed for implantation in the body of the patient, especially in the spinal cord region. Suitable delivery systems are described in U.S. Pat. No. 5,550,050 and published PCT Application Nos. WO 92/19195, WO 94/25503, WO 95/01203, WO 95/05452, WO 96/02286, WO 96/02646, WO 96/40871, WO 96/40959 and WO 97/12635. Suitable DNA sequences can be prepared synthetically for each active agent on the basis of the developed sequences and the known genetic code.

The active agent is preferably administered in a therapeutically effective amount. By a “therapeutically effective amount” or simply “effective amount” of an active compound is meant a sufficient amount of the compound to treat the desired condition at a reasonable benefit/risk ratio applicable to any medical treatment. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington: The Science and Practice of Pharmacy.

Dosage may be adjusted appropriately to achieve desired drug levels, locally or systemically. Typically the active agents of the present invention exhibit their effect at a dosage range from about 0.001 mg/kg to about 250 mg/kg, preferably from about 0.01 mg/kg to about 100 mg/kg of the active ingredient, more preferably from a bout 0.05 mg/kg to about 75 mg/kg. A suitable dose can be administered in multiple sub-doses per day. Typically, a dose or sub-dose may contain from about 0.1 mg to about 500 mg of the active ingredient per unit dosage form. A more preferred dosage will contain from about 0.5 mg to about 100 mg of active ingredient per unit dosage form. Dosages are generally initiated at lower levels and increased until desired effects are achieved. In the event that the response in a subject is insufficient at such doses, even higher doses (or effective higher doses by a different, more localized delivery route) may be employed to the extent that patient tolerance permits. Continuous dosing over, for example, 24 hours or multiple doses per day are contemplated to achieve appropriate systemic levels of compounds.

Advantageously, the compositions are formulated as dosage units, each unit being adapted to supply a fixed dose of active ingredients. Tablets, coated tablets, capsules, ampoules and suppositories are examples of dosage forms according to the invention.

It is only necessary that the active ingredient constitute an effective amount, i.e., such that a suitable effective dosage will be consistent with the dosage form employed in single or multiple unit doses. The exact individual dosages, as well as daily dosages, are determined according to standard medical principles under the direction of a physician or veterinarian for use humans or animals.

The compositions will generally contain from about 0.0001 to 99 wt. %, preferably about 0.001 to 50 wt. %, more preferably about 0.01 to 10 wt. % of the active ingredient by weight of the total composition. In addition to the active agent, the compositions and medicaments can also contain other pharmaceutically active compounds. Examples of other pharmaceutically active compounds include, but are not limited to, analgesic agents, cytokines and therapeutic agents in all of the major areas of clinical medicine. When used with other pharmaceutically active compounds, the conopeptides of the present invention may be delivered in the form of drug cocktails. A cocktail is a mixture of any one of the compounds useful with this invention with another drug or agent. In this embodiment, a common administration vehicle (e.g., pill, tablet, implant, pump, injectable solution, etc.) would contain both the instant composition in combination with a supplementary potentiating agent. The individual drugs of the cocktail are each administered in therapeutically effective amounts. A therapeutically effective amount will be determined by the parameters described above; but, in any event, is that amount which establishes a level of the drugs in the area of body where the drugs are required for a period of time which is effective in attaining the desired effects.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., Molecular Cloning (Cold Spring Harbor Laboratory Press, Cold Spring Harbor, N.Y., 1982); Sambrook et al., Molecular Cloning, 2nd Ed. (Cold Spring Harbor Laboratory Press, Cold Spring Harbor, N.Y., 1989); Sambrook and Russell, Molecular Cloning, 3rd Ed. (Cold Spring Harbor Laboratory Press, Cold Spring Harbor, N.Y., 2001); Ausubel et al., Current Protocols in Molecular Biology (John Wiley & Sons, updated through 2005); Glover, DNA Cloning (IRL Press, Oxford, 1985); Anand, Techniques for the Analysis of Complex Genomes, (Academic Press, New York, 1992); Guthrie and Fink, Guide to Yeast Genetics and Molecular Biology (Academic Press, New York, 1991); Harlow and Lane, Antibodies, (Cold Spring Harbor Laboratory Press, Cold Spring Harbor, N.Y., 1998); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, (Blackwell Scientific Publications, Oxford, 1988); Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), 4th Ed., (Univ. of Oregon Press, Eugene, Oreg., 2000).

### EXAMPLES

The present invention can be described by reference to the following Examples, which are offered by way of illustration and are not intended to limit the invention in any manner. Standard techniques well known in the art or the techniques specifically described below were utilized.

### Example 1

### Experimental Procedures

Extraction and fractionation of Conus planorbis venom: Snails were collected in Cebu and Marinduque Islands, Philippines and dissected. Venom was pressed out of freshly dissected venom ducts that were kept on ice. Venom pooled from several snails was lyophilized and stored at −70° C. A 500-mg portion was resuspended in 35 ml of 30% acetonitrile-0.2% trifluoroacetic acid (TFA) using a vortex mixer for 2×1 min with an interval of 5 min on ice. The mixture was sonicated using a Branson LS-75 probe for 3×0.5 min on ice with 1-min rest periods, and the sediment was pelleted in a Beckman Avanti centrifuge with an F650 rotor. Centrifugation was done for 30 min at 37,500×g. The supernatant was diluted with 0.1% TFA, centrifuged again to remove all residual particles, and applied to a preparative Vydac C₁₈ high-pressure liquid chromatography (HPLC) column (2.5 cm×25 cm). Venom peptides were eluted from the column with a linear gradient of 4.5% to 90% acetonitrile-0.1% TFA at 0.9% acetonitrile/min. The flow rate was 20 ml/min, and the absorbance of the eluate was monitored at 220 nm. An analytical Vydac C₁₈ HPLC column (4.6 mm by 250 mm) with a linear gradient at 0.45% acetonitrile/min in 0.1% TFA at a flow rate of 1 ml/min was used for subfractionation.

Bioassay of HPLC fractions on Nireis virens: Specimens (usually within 10-16 cm long) of N. virens were maintained in seawater at 0-4° C. in the cold room. During injections, the worms were taken outside the cold room in individual beakers containing cold seawater. The temperature was maintained close to 4° C. by keeping the beakers immersed in iced water. Each worm was laid out on a block of styrofoam right before injection and returned to cold seawater within a few seconds after injection. Lyophilized samples resuspended in 10-12 μl of normal saline solution (NSS) were injected using an insulin syringe at the anterior end of the worm. A needle lid with the tip cut off was used to obtain a consistent depth of needle puncture of ˜1.5 mm. The initial effects of injections were evaluated by transferring the worms to seawater at room temperature for 5-15 sec at approximately 1 h after injection. These were observed side by side with saline-injected controls continuously for at least 4 h and checked every day for at least 2 weeks.

Characterization of peptides: Samples were completely reduced with 10 mM dithiothreitol at 65° C. for 15 minutes and completely alkylated using 0.7% 4-vinylpyridine for 20 min in the dark at room temperature. Alkylation reactions were diluted 10-fold with 0.1% TFA and fractionated in an analytical C₁₈ HPLC column. Elution was done with a linear gradient of 1.8% acetonitrile/min in 0.1% TFA at a flow rate of 1 ml/min.

Peptide sequencing by Edman degradation chemistry was done on alkylated samples after HPLC purification. Uncertain readings of C-terminal residues were verified by sequencing of fragments obtained after endopeptidase digestion with Arg-C. The reaction conditions for enzyme digestion were as recommended by the supplier (Roche Diagnostics Corporation, Indianapolis, Ind.).

Mass determinations on both native and synthetic peptides were done by matrix-assisted laser desorption ionization (MALDI) or electrospray ionization (ESI) mass spectrometry (MS) at the University of Utah Mass Spectrometry and Proteomic Core Facility, the Salk Institute Peptide Biology Lab, and The University of Queensland Institute for Molecular Bioscience.

Chemical synthesis and purification of CPY-Pl1 and CPY-Fe1: CPY-Pl1 and CPY-Fe1 were synthesized on an ABI 433 peptide synthesizer (Applied Biosystems, Foster City, Calif.) following the “Fastmoc” chemistry procedure from ABI. Standard protecting groups for the amino acid side chains were used. The cleaved peptide was further purified by HPLC on a semi-prep C₁₈ column with a linear gradient of 0.18% acetonitrile/min in 0.1% TFA. The flow rate was 2.5 ml/min, and absorbance at 220 and 280 nm was monitored.

NMR spectroscopy: Samples for ¹H NMR measurements contained ˜2 mM peptide in 90% H₂O/10% D₂O (v/v) at ˜pH 3 or 20% TFE/80% H₂O/10% D₂O, Spectra were recorded at 288-298 K on a Bruker ARX-600 spectrometer equipped with a shielded gradient unit. 2D NMR spectra were recorded in phase-sensitive mode using time-proportional phase incrementation for quadrature detection in the t₁ dimension as previously described for other disulfide-rich peptides (10,11).

Spectra were processed on a Silicon Graphics Indigo workstation using XWINNMR (Bruker) software. The tᵢ dimension was zero-filled to 1024 real data points, and 90° phase-shifted sine bell window functions were applied prior to Fourier transformation. Chemical shifts were referenced to internal 2,2-dimethyl-2-silapentane-5-sulfonate. NMR spectral assignments for CPY-Pl1 were made using established techniques (12).

Bioassay of synthetic samples in mice and C. elegans: Intracranial injections were made in mice (Swiss-Webster) that were 15-17 days old. Peptide samples in HPLC solutions were lyophilized, resuspended in 12 μl of NSS, and administered intracranially to mice using an insulin syringe. The peptide-injected mice were observed side by side with saline-injected controls continuously for 2-4 h, and checked the next day.

Microinjection of peptide samples into the pseudocoelomic region of the nematode Cenorhabditis elegans was done as described (13).

K⁺ channel assays: The Xenopus oocyte expression system was used to study the effect of the CPY peptides on K⁺ and Na⁺ channels. Oocytes were treated, and channels were expressed as described (14). Whole-cell currents were recorded at room temperature (19-22° C.) under two-electrode voltage-clamp control using an 0C-725C amplifier (Warner Instruments, Hamden, Conn.) or a Turbo-Tec amplifier (npi electronic, Tamm, Germany). Current records were low-pass filtered at 3 kHz and sampled at 10 kHz, or at 1 kHz (−3 db) and sampled at 4 kHz. The bath solution was normal frog Ringer's (15) containing 115 mM NaCl, 2.5 mM KCl, 1.8 mM CaCl₂ and 10 mM Hepes (pH 7.2) (NaOH). Bovine serum albumin (0.1 mg/ml) was added to the bath solution in all the dose-response experiments.

The IC₅₀ values for the block of Kv1.6 channel were calculated from the peak currents at a test potential of 20 mV or 0 mV according to IC₅₀=fc/(1−fc)×[Tx], where fc is the fractional current and [Tx] is the toxin concentration.

Cloning—Total RNA preparations from a single duct each of Conus planorbis and Conus ferrugineus (FIG. 1) were obtained using the Qiagen RNeasy mini (Qiagen Sciences, Valencia, Calif.) protocol for isolation of total RNA from animal tissues. Each RNA preparation (4.5 μg for C. planorbis and 5.1 μg for C. ferrugineus) was used in cDNA synthesis (16). Degenerate primers derived from CPY-Pl1 and CPY-Fe1 sequences were utilized in 3′RACE (16) and 5′RACE (SMART RACE, Clontech Laboratories Inc., Mountain View, Calif.) experiments on C. planorbis cDNA. Oligonucleotide primers designed from the signal sequence identified for CPY-Pl1 were used in a 3′RACE (16) on C. planorbis and C. ferrugineus cDNA. All PCR runs were done in a PTC-200 Peltier Thermal Cycler (MJ Research, Inc., Waltham, Mass.) using High Fidelity Platinum Taq DNA polymerase (Invitrogen Corporation, Carlsbad, Calif.). The TOPO TA cloning kit (Invitrogen Corporation, Carlsbad, Calif.) was used for all transformations. DNA sequencing was done at the University of Utah Huntsman Cancer Institute Protein/DNA Core Facility using samples prepared following the Qiagen mini prep (Qiagen Sciences, Valencia, Calif.) kit protocol.

### Example 2

### Isolation of CPY Peptides from the Venom of C. planorbis (9)

Crude Conus planorbis venom was fractionated by HPLC as described in Experimental Procedures. Biological activity on the marine worm N. virens was used to identify active fractions from the preparative HPLC column (FIG. 1A) and guide further subfractionation (FIG. 1B). Aliquots of the fractions and subfractions were pooled, lyophilized and assayed on N. virens as mentioned in Experimental Procedures; if a pool showed bioactivity as described below, individual fractions were assayed. The fraction indicated in FIG. 1A gave characteristic symptoms when injected into N. virens. Worms that had not been injected, or those injected with the control solution (NSS) would move in energetic S-like patterns when transferred to seawater at room temperature. In contrast, affected worms did not move in S-like patterns, but would just glide or move very slowly. All injected worms were moved back to cold seawater after 5-15 seconds of observation in seawater at room temperature; worms that manifested the initial symptomatology eventually became weak or died in 1-5 days. Uninjected and control worms survived for months.

The subfractions in FIG. 1B were assayed individually, and one peak exhibited the activity on N. virens described above. The active peak in FIG. 1B was completely reduced and alkylated as described in Experimental Procedures. The reduced and alkylated material was fractionated by HPLC (FIG. 1C); the earlier-eluting peak indicated by the arrow is CPY-Pl1 and the later-eluting peak is CPY-Fe1.

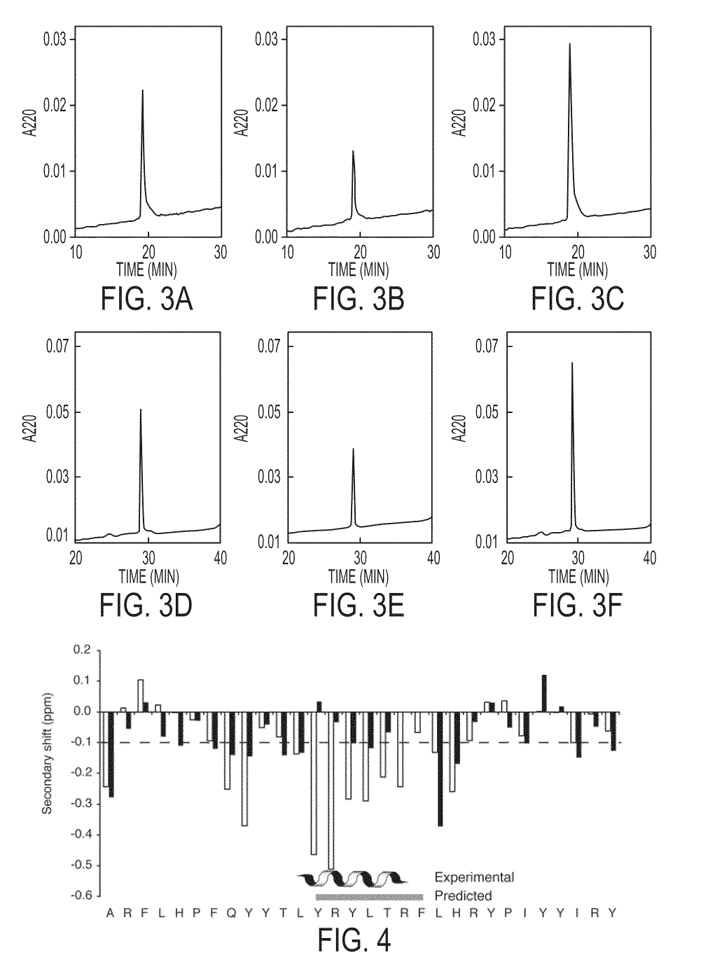

The peptides were sequenced using standard Edman methods. The peptide sequences obtained had a most striking sequence feature: an unusually high frequency of tyrosine residues, in addition to basic and hydrophobic residues. The sequences of the two peptides are shown in FIG. 3; the monoisotopic masses of 4092.1 and 4040.1 Da that were obtained corresponded to the respective calculated masses for CPY-Pl1 and CPY-Fe1. Sixteen out of the thirty residues are identical in the two peptides, including five tyrosine residues.

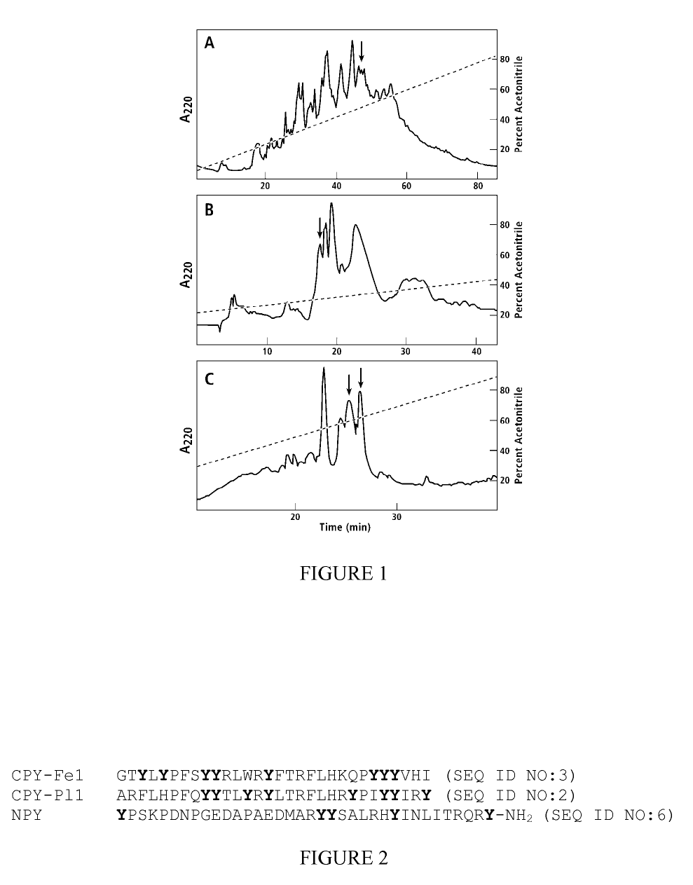

The high content of tyrosine in CPY-Pl1 and CPY-Fe1 is reminiscent of the vertebrate neuropeptide Y family (NPY) (see FIG. 2); we refer to these peptides as the conopeptide Y (CPY) family. We propose to name these CPY-family peptides from Conus venoms using the following nomenclature; the individual peptides will be indicated by the first two letters of the species name, followed by a number representing the numerical sequence of peptides that belong to this family from each species. The two Y-rich peptides isolated from what has been presumed to be C. planorbis venom are referred to as CPY-Fe1 (Fe for ferrugineus) and CPY-Pl1 (Pl for planorbis). Results from cloning, described below, suggest that both of C. ferrugineus and C. planorbis venoms were present in the sample labeled “pooled C. planorbis venom” that was used. Due to the close resemblance of shells of the two species, the mistaken identification of one species for the other was highly probable during the period that specimens were accumulated to prepare the pooled crude venom that yielded the purified peptides.

### Example 3

### Chemical Synthesis of CPY Peptides

The chemical synthesis of the CPY peptides was carried out as described under Experimental Procedures. The HPLC chromatograms for the coelution of synthetic CPY-Fe1 and CPY-Pl1 with the respective native samples are shown in FIGS. 3A (9) and 3B. The isotopic peaks in the mass spectra of both synthetic peptides matched those in the spectra of the native samples.

### Example 4

### NMR Spectroscopy

The secondary shifts of CPY-Pl1 in aqueous solution and in 20% TFE are shown in FIG. 4. In general, analysis of aH chemical shifts of peptides can provide a strong indication of the type of secondary structure present. For CPY-Pl1 in aqueous solution the chemical shifts suggest that no regular secondary structure is present based on the lack of stretches of consistent positive or negative secondary shifts. However, in the presence of TFE a series of significant (i.e., >0.1 ppm) negative deviations from random coil shifts between residues 12 to 18 suggests the presence of helical structure in this region. TFE is commonly reported to stabilize helical structures in peptides having an intrinsic helical tendency and this appears to be the case for CPY-Pl1.

### Example 5

### Bioassay of CPY-Fe1 Using Mice and C. elegans

Mice were injected intracranially with CPY-Fe1. Initial observations on mice injected with approximately 0.7 nmol of peptide per gram of mouse only gave mild symptoms such as slightly more than usual grooming, climbing or hyperactivity symptoms. An attempt to elicit more definitive symptomatology by dropping the metal lid onto the cage, thereby creating a loud acoustic input, caused a 30-second seizure in a mouse injected with ˜1.5 nmol of peptide per gram of mouse. A surprising effect was elicited when one peptide-injected mouse was accidentally dropped while being transferred between two cages; not only was a seizure elicited, but death occurred within 10-15 seconds after the seizure was initiated. The seizures were observed within 2-3 min after the drop. Thus, we routinely carried out a test referred to as “the mouse-drop assay” after all succeeding mouse intracranial injections of the CPY peptides. Injected mice were dropped from a height of approximately 1.5 ft on to the cage bottom starting at ˜3-5 min after injection. This was repeated every 5-10 min up to ˜30 min after injection. The seizure response to the mouse drop is usually observed after the first drop. No symptoms were observed in NSS-injected controls or unaffected mice, even after 4-6 drops within the 30-min period after injection. Both peptides induced seizures with the mouse-drop assay. The dose-response was determined for the CPY-Fe1 peptide. At a dose of 5 nmol, approximately 50% of the mice responded with mild to moderate seizures. With 20 nmol injected per mouse, most of the mice developed severe seizures and died.

Microinjections in C. elegans were made at a dose of ˜10 pmol of CPY-Fe1 per worm. Partial paralysis, which lasted for ˜20 min, was observed in 4 out of 4 worms injected. In 3 out of the 4 worms, paralysis occurred from the midsection to the posterior end; the fourth worm showed paralysis from midsection to the anterior end.

An initial test on the effect of CPY-Fe1 on the biphasic contractile response of electrically stimulated rat prostatic vas deferens (17) did not show any effect on both noradrenaline- and ATP-mediated responses with 10 μM CPY-Fe1.

### Example 6

### Electrophysiology

Initial screening for activity using channels expressed in Xenopus oocytes included Kv1.1 to Kv1.6, Kv2.1, Kv3.4 and Nav1.2. In these experiments, the oocytes used were free of the vitelline membrane (“skinned”) during the clamping experiments. CPY-Fe1 was found to be active at 1 μM on Kv1.6. When attempts were made to determine IC₅₀ values with skinned oocytes, evidence for high affinity block of Kv1.6 was obtained for both peptides. However, there were technical problems using skinned oocytes since these invariably developed a leak current after a variable length of time as the measurements were being made. Thus, it was not feasible to obtain reproducible quantitative measurements using skinned oocytes.

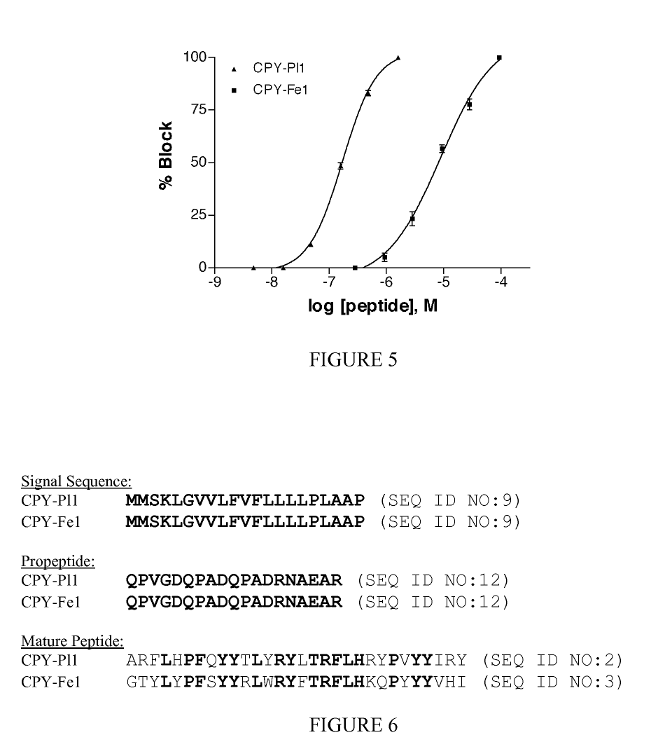

The dose-response for both CP-Fe1 and CPY-Pl1 (FIG. 5) demonstrates that CPY-Pl1 is more potent on the Kv1.6 subtype. The data shown in Table 2 and FIG. 5 were obtained using unskinned Xenopus oocytes; the kinetics were slow and took tens of minutes to reach equilibrium. Table 2 presents the IC₅₀ values obtained from dose-response experiments in oocytes expressing Kv1.2 to Kv1.6. CPY-Pl1 is more active than CPY-Fe1 on both Kv1.2 (>15-fold) and Kv1.6 (˜50-fold).

TABLE 2
Activity of CPY Peptides on Mammalian
Kv1 Isoforms Expressed in Xenopus Oocytes

| | IC₅₀, μM (95% Confidence Interval) | | | | |
|---|---|---|---|---|---|
| Peptide | Kv1.2 | Kv1.3 | Kv1.4 | Kv1.5 | Kv1.6 |
| CPY-Fe1 | >30 | >50 | >50 | >50 | 8.8 (6.5-12.0) |
| CPY-Pl1 | 2.0 (1.0-3.9) | >50 | >50 | >50 | 0.17 (0.16-0.19) |

### Example 7

### Molecular Cloning Definition of the CPY Family

A cDNA library made from Conus planorbis venom ducts was used to identify the precursors of the CPY peptides. The predicted open reading frame including signal sequence, propeptide and mature peptide is shown in FIG. 6. The cleavage site between the signal peptide, MMSKLGVVLFVFLLLLPLAAP (SEQ ID NO:9, and the propeptide (SEQ ID NO:12) was predicted through SignalP 3.0 server (http colon slash slash dot cbs dot dtu dot dk slash services slash SignalP slash).

Initial 3′RACE experiments utilizing C. planorbis cDNA and oligonucleotide primers derived from both peptides yielded clones (7/7) for CPY-Pl1 but no clones for CPY-Fe1. Clones (6/6) obtained in 5′RACE experiments for both CPY peptides on C. planorbis cDNA using primers from both peptides gave the CPY-Pl1 precursor even with the primer derived from CPY-Fe1, which has a region homologous with the primer derived from CPY-Pl1. A 3′RACE experiment on C. planorbis cDNA utilizing oligonucleotide primers derived from the signal sequence gave CPY-Pl1 clones (7/7) up to the polyA tail. A screen for CPY peptides in C. ferrugineus cDNA using one of the primers derived from the signal sequence and the polyT-containing Q_{T} (16) primer gave clones (4/4) for CPY-Fe1.

The signal and propeptide sequences are completely shared by the two peptides up to the nucleotide level. Sixteen (FIG. 6) out of the 30 residues of the mature peptide are also shared up to the nucleotide level. There is 91% homology in the 3′UTR regions of CPY-Fe1 (EU000529) and CPY-Pl1 (EU000528). The unique signal sequence revealed from clones encoding precursors of these peptides defines a new gene family of conopeptides, which we refer to as the Conopeptide Y or CPY family.

The peptides characterized herein are unusual in their high content of tyrosine, normally an uncommon amino acid. Most Conus venom peptides are stabilized by multiple disulfide bonds. As a rule, the Conus venom peptides that lack disulfide cross-links tend to be smaller in size than the disulfide-rich conotoxins. The CPY peptides that are characterized here are the longest conopeptides without disulfides shown to be biologically active. In addition to the high tyrosine content, these peptides are enriched in arginine and large hydrophobic residues (F, L and I); in CPY-Pl1, these five amino acids account for over 70% of the total.

Both CPY-Fe1 and CPY-Pl1 were isolated (9) from a batch of venom, which had been assumed to have come from specimens of C. planorbis. The cloning results, however, strongly indicated that one of the CPY peptides that was isolated from the pooled venom, CPY-Fe1, could be cloned only from cDNA derived from a C. ferrugineus venom duct. Correspondingly, only CPY-Pl1 clones (20/20) were detected from C. planorbis cDNA. There are shells of C. ferrugineus or C. planorbis that could easily be confused as one or the other, indeed, the precise taxonomy of the complex of species related to Conus planorbis remains unresolved. Each cDNA preparation was obtained from a single venom duct of each species; thus, the cloning results seem more reliable than extraction from venom pooled from a hundred or so specimens.

We demonstrated that these peptides are antagonists of voltage-gated K⁺ channels belonging to the Kv1 subfamily. The variable onset of leak currents in skinned oocytes suggests that in addition to the K⁺ channel blocking activity, the CPY peptides may also be membrane-active. Since these are hydrophobic peptides, the possibility that they can insert into membranes to form pores or destabilize the oocyte membranes in some other manner could explain the unpredictable leak observed; in contrast, the unskinned oocytes remained stable enough so that the reliable dose response data shown in FIG. 5 and Table 2 could be obtained. A small peptide lacking disulfides that inhibited voltage-gated K⁺ channels was previously reported from C. monile; the subfamily selectivity was not reported (18). The CPY peptide that has been most well characterized, CPY-Pl1, is highly selective for the Kv1.6 subtype. It is notable that CPY-Pl1 is ˜18-fold more potent on Kv1.6 than pl14a, a disulfide-rich peptide that was previously isolated from the same venom (9,19).

Conus species have a structurally diverse group of peptides targeted to the Kv1 subfamily; the first such peptide characterized was κ-conotoxin PVIIA (20), from the venom of the fish-hunting cone snail Conus purpurascens; this peptide has three disulfide bonds and belongs to the O-gene superfamily. Surprisingly, when Kv1 channel-targeted peptides from two other clades of fish-hunting cones were elucidated, peptides structurally unrelated to κ-conotoxin PVIIA were found: κM-conotoxin RIIIK (21) from Conus radiatus also has three disulfide bonds, but belongs to the M-gene superfamily and conkunitzin S1 (22,23) is a Kunitz-domain K⁺ channel inhibitor from Conus striatus. The investigation of Conus planorbis venom not only revealed K⁺ channel targeted peptides unrelated to the three classes above, but two different toxins unrelated to each other that both preferentially target Kv1.6: κJ-conotoxin PIXIVA, a peptide with two disulfide bonds and previously called pl14a (9,19), and CPY-Pl1, the peptide characterized in this report. It is not unprecedented for venom from one species to contain unrelated peptides that both antagonize the same ion channel complex. It has previously been shown that different peptides antagonize the same nicotinic receptor subtype in both Conus purpurascens (24,25), and Conus imperialis venoms (26). In both cases, the peptides were structurally unrelated and were subsequently shown to target different pharmacological sites on the same receptor complex. This appears to be part of the sophisticated combination drug therapy strategy that cone snails employ for prey paralysis. We speculate that the natural prey of Conus planorbis has a Kv1.6-like Shaker-type K⁺ channel that may be an important molecular target in the snail's strategy for prey capture.

Multiple disulfide cross-links presumably stabilize most Conus peptides once they are injected into the body of the targeted animal. Since the CPY peptides are unstructured in aqueous solution, they would appear to be subject to rapid proteolytic degradation. However, a significant number of Conus peptide toxins may target axons right at the site of injection; in the case of fish-hunting cone snails, K⁺ channel blockers are part of the “lightning-strike cabal” of toxins that cause a massive depolarization of axons in the immediate vicinity of the injection site, which in effect rapidly immobilizes the prey. The CPY peptides in Conus planorbis and Conus ferrugineus venoms may play an analogous role in vivo. The peptides may act right at the injection site, and once bound to their targets, would become much more highly structured and resistant to attack by proteases.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

It will be appreciated that the methods and compositions of the instant invention can be incorporated in the form of a variety of embodiments, only a few of which are disclosed herein. Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### BIBLIOGRAPHY

1. Terlau, H., and Olivera, B. M. (2004) Physiol. Rev. 84, 41-68.
2. Craig, A. G., Norberg, T., Griffin, D., Hoeger, C., Akhtar, M., Schmidt, K., Low, W., Dykert, J., Richelson, E., Navarro, V., Macella, J., Watkins, M., Hillyard, D., Imperial, J., Cruz, L. J., and Olivera, B. M. (1999) J. Biol. Chem. 274, 13752-13759.
3. Olivera, B. M., McIntosh, J. M., Clark, C., Middlemas, D., Gray, W. R., and Cruz, L. J. (1985) Toxicon 23, 277-282.
4. Maillo, M., Aguilar, M. B., Lopez-Vera, E., Craig, A. G., Bulaj, G., Olivera, B. M., and Heimer de la Cortera, E. P. (2002) Toxicon 40, 401-407.
5. Cruz, L. J., de Santos, V., Zafaralla, G. C., Ramilo, C. A., Zeikus, R., Gray, W. R., and Olivera, B. M. (1987) J. Biol. Chem. 262, 15821-15824.
6. Jimenez, E. C., Olivera, B. M., Gray, W. R., and Cruz, L. J. (1996) J. Biol. Chem. 281, 28002-28005.
7. Espiritu, D. J. D., Watkins, M., Dia-Monje, V., Cartier, G. E., Cruz, L. J., and Olivera, B. M. (2001) Toxicon 39, 1899-1916.
8. Duda Jr, T. F., Kohn, A. J., and Palumbi, S. R. (2001) Biol. J. Linnean Soc. 73, 391-409.
9. Imperial, J. S. (2007) Novel peptides from Conus planorbis, Terebra subulata, and Hastula hectica (Ph. D. dissertation). In., The University of Queensland, Brisbane.
10. Daly, N. L., Ekberg, J. A., Thomas, L., Adams, D. J., Lewis, R. J., and Craik, D. J. (2004) J. Biol. Chem. 279, 25774-25782.
11. Rosengren, K. J., Daly, N. L., Plan, M. R., Waine, C., and Craik, D. J. (2003) J. Biol. Chem. 278, 8606-8616.
12. Wüthrich, K. (1986) NMR of Proteins and Nucleic Acids, Wiley-Interscience, New York.
13. Mello, C. C., Kramer, J. M., Stirchcomb, D., and Ambros, V. (1991) EMBO J. 10, 2959-3970.
14. Jacobsen, R. B., Koch, E. D., Lang-Malecki, B., Stocker, M., Verhey, J., van Wagoner, R. M., Vyazovkina, A., Olivera, B. M., and Terlau, H. (2000) J. Biol. Chem. 275, 24639-24644.
15. Horton, R. M., Manfredi, A. A., and Conti-Tronconi, B. M. (1993) Neurology 43, 983-986.
16. Frohman, M. A. (1990) RACE: rapid amplification of cDNA ends. In: Innis, M. A. (ed). PCR Protocols, Academic Press, San Diego, Calif.
17. Sharpe, I. A., Gehrmann, J., Loughnan, M. L., Thomas, L., Adams, D. A., Atkins, A., Palant, E., Craik, D. J., Alewood, P. F., and Lewis, R. J. (2001) Nature Neuroscience 4(9), 902-907.
18. Sudarslal, S., Singaravadivelan, G., Ramasamy, P., Ananda, K., Sarma, S. P., Sikdar, S. K., Krishnan, K. S., and P., B. (2004) Biochem. Biophys. Res. Commun. 317, 682-688.
19. Imperial, J. S., Bansal, P. S., Alewood, P. F., Daly, N. L., Craik, D. J., Sporning, A., Terlau, H., Lopez-Vera, E., Bandyopadhyay, P. K., and Olivera, B. M. (2006) Biochemistry 45(27), 8331-8340.
20. Shon, K., Stocker, M., Terlau, H., Stühmer, W., Jacobsen, R., Walker, C., Grilley, M., Watkins, M., Hillyard, D. R., Gray, W. R., and Olivera, B. M. (1998) J. Biol. Chem. 273, 33-38.
21. Al-Sabi, A., Lennartz, D., Ferber, M., Gulyas, J., Rivier, J. E., Olivera, B. M., Carlomagno, T., and Terlau, H. (2004) Biochemistry 43, 8625-8635.
22. Imperial, J. S., Silverton, N., Olivera, B. M., Bandyopadhyay, P. K., Sporning, A., Ferber, M., and Terlau, H. (2007) Proceedings of the American Philosophical Society 151, 185-200.
23. Bayrhuber, M., Vijayan, V., Ferber, M., Graf, R., Korukottu, J., Imperial, J., Garrett, J. E., Olivera, B. M., Terlau, H., Zweckstetter, M., and Becker, S. (2005) J. Biol. Chem. 180, 21246-21255.
24. Hopkins, C., Grilley, M., Miller, C., Shon, K., Cruz, L. J., Gray, W. R., Dykert, J., Rivier, J., Yoshikami, D., and Olivera, B. M. (1995) J. Biol. Chem. 270, 22361-22367.
25. Shon, K., Grilley, M., Jacobsen, R., Cartier, G. E., Hopkins, C., Gray, W. R., Watkins, M., Hillyard, D. R., Rivier, J., Tones, J., Yoshikami, D., and Olivera, B. M. (1997) Biochemistry 36, 9581-9587.
26. Ellison, M., McIntosh, J. M., and Olivera, B. M. (2003) J. Biol. Chem. 278, 757-764.
27. Wishart, D. S., Bigam, C. G., Hodges, R. S., and Sykes, B. D. (1995) J. Biomol. NMR 5, 67-81.
28. McIntosh, J. M. et al. (1998) Methods Enzymol 294, 605-624.
29. Van de Steen, P. et al. (1998) Critical Rev. in Biochem. and Mol. Biol. 33, 151-208.
30. Sambrook, J. et al. (1989) Molecular Cloning: A Laboratory Manual, 2nd Ed., Cold Spring Harbor Laboratory, Cold Spring Harbor, N.Y.
31. Hammerland, L. G. et al. (1992). Eur. J. Pharmacol. 226:239-244.

What is claimed is:

1. An isolated peptide selected from the group consisting of
(a) a CPY peptide having the generic forumula Xaa1-Xaa2-Xaa3-Leu-Xaa5-Pro-Phe-Xaa8-Tyr-Tyr-Xaa11-Leu-Xaa13-Arg-Tyr-Xaa16-Thr-Arg-Phe-Leu-His-Xaa22-Xaa23-Pro-Xaa25-Tyr-Tyr-Xaa28-Xaa29-Xaa30 (SEQ ID NO:1),
wherein Xaa1 is Gly, Ala, an aliphatic amino acids bearing linear or branched saturated hydrocarbon chains such as Leu (D or L), Ile and Val or non-natural derivatives of the aliphatic amino acid; Xaa2 is Thr, g-Thr (where g is glycosylation), Ser, g-Ser, Arg, Lys, ornithine, homo-Lys, homoarginine, nor-Lys, N-methyl-Lys, N,N′-dimethyl-Lys, N,N′,N″-trimethyl-Lys or any synthetic basic amino acid; Xaa3 is Phe, Tyr, meta-Tyr, ortho-Tyr, nor-Tyr, mono-halo-Tyr, di-halo-Tyr, O-sulpho-Tyr, O-phospho-Tyr, nitro-Tyr; Xaa5 is His, Tyr, meta-Tyr, ortho-Tyr, nor-Tyr, mono-halo-Tyr, di-halo-Tyr, O-sulpho-Tyr, O-phospho-Tyr, nitro-Tyr; Xaa8 is Ser, g-Ser, Thr, g-Thr, Gln, Asn Xaa11 is Thr, g-Thr, Ser, g-Ser, Arg, Lys, ornithine, homo-Lys, homoarginine, nor-Lys, N-methyl-Lys, N,N′-dimethyl-Lys, N,N′,N″-trimethyl-Lys or any synthetic basic amino acid; Xaa13 is Trp (D or L), halo-Trp (D or L), Tyr, meta-Tyr, ortho-Tyr, nor-Tyr, mono-halo-Tyr, di-halo-Tyr, O-sulpho-Tyr, O-phospho-Tyr, nitro-Tyr; Xaa16 is Phe, an aliphatic amino acids bearing linear or branched saturated hydrocarbon chains such as Leu (D or L), Ile and Val or non-natural derivatives of the aliphatic amino acid; Xaa22 is Lys, Arg, ornithine, homo-Lys, homoarginine, nor-Lys, N-methyl-Lys, N,N′-dimethyl-Lys, N,N′,N″-trimethyl-Lys or any synthetic basic amino acid; Xaa23 is Gln, Asn, Tyr, meta-Tyr, ortho-Tyr, nor-Tyr, mono-halo-Tyr, di-halo-Tyr, O-sulpho-Tyr, O-phospho-Tyr, nitro-Tyr; Xaa25 is an aliphatic amino acids bearing linear or branched saturated hydrocarbon chains such as Leu (D or L), Ile and Val or non-natural derivatives of the aliphatic amino acid, Tyr, meta-Tyr, ortho-Tyr, nor-Tyr, mono-halo-Tyr, di-halo-Tyr, O-sulpho-Tyr, O-phospho-Tyr, nitro-Tyr; Xaa28 is an aliphatic amino acids bearing linear or branched saturated hydrocarbon chains such as Leu (D or L), Ile and Val or non-natural derivatives of the aliphatic amino acid; Xaa29 is His, Arg, Lys, ornithine, homo-Lys, homoarginine, nor-Lys, N-methyl-Lys, N,N′-dimethyl-Lys, N,N′,N″-trimethyl-Lys or any synthetic basic amino acid; and Xaa30 is an aliphatic amino acids bearing linear or branched saturated hydrocarbon chains such as Leu (D or L), Ile and Val or non-natural derivatives of the aliphatic amino acid, Tyr, meta-Tyr, ortho-Tyr, nor-Tyr, mono-halo-Tyr, di-halo-Tyr, O-sulpho-Tyr, O-phospho-Tyr, nitro-Tyr; and
(b) a derivative of (a), wherein the derivative is the peptide (a) in which the Pro residues may be substituted with hydroxyl-Pro; the Arg residues may be substituted by Lys, ornithine, homoargine, nor-Lys, N-methyl-Lys, N,N-dimethyl-Lys, N,N,N-trimethyl-Lys or any synthetic basic amino acid; the Lys residues may be substituted by Arg, ornithine, homoargine, nor-Lys, or any synthetic basic amino acid; the Tyr residues may be substituted with any synthetic hydroxy containing amino acid; the Ser residues may be substituted with Thr or any synthetic hydroxylated amino acid; the Thr residues may be substituted with Ser or any synthetic hydroxylated amino acid; the Phe and Trp residues may be substituted with any synthetic aromatic amino acid; and the Asn, Ser, Thr or Hyp residues may be glycosylated; the Tyr residues may also be substituted with the 3-hydroxyl or 2-hydroxyl isomers (meta-Tyr or ortho-Tyr, respectively) and corresponding O-sulpho- and O-phospho-derivatives; the aliphatic amino acids may be substituted with one another or with Ala or with synthetic derivatives bearing non-natural aliphatic branched or linear side chains CₙH₂ₙ₊₂ up to and including n=8; the Leu residues may be substituted with Leu (D) and the Trp residues may be substituted with Trp (D).

2. The isolated peptide of claim 1 which is selected from the group consisting of SEQ ID NO:2 and SEQ ID NO:3.

3. The isolated peptide of claim 1 which is a derivative of the peptide of (a) selected from the group consisting of SEQ ID NO:2 and SEQ ID NO:3.

4. The isolated peptide of claim 1, which is modified to contain an O-glycan, an S-glycan or an N-glycan.

5. The isolated peptide of claim 2, which is modified to contain an O-glycan, an S-glycan or an N-glycan.

6. The isolated peptide of claim 3, which is modified to contain an O-glycan, an S-glycan or an N-glycan.

7. A composition comprising a peptide of claim 1 or a pharmaceutically acceptable salt or solvate thereof and a pharmaceutically acceptable carrier.

8. A composition comprising a peptide of claim 2 or a pharmaceutically acceptable salt or solvate thereof and a pharmaceutically acceptable carrier.

9. A composition comprising a peptide of claim 3 or a pharmaceutically acceptable salt or solvate thereof and a pharmaceutically acceptable carrier.

10. A composition comprising a peptide of claim 4 or a pharmaceutically acceptable salt or solvate thereof and a pharmaceutically acceptable carrier.

11. A composition comprising a peptide of claim 5 or a pharmaceutically acceptable salt or solvate thereof and a pharmaceutically acceptable carrier.

12. A composition comprising a peptide of claim 6 or a pharmaceutically acceptable salt or solvate thereof and a pharmaceutically acceptable carrier.

13. A method of identifying compounds that mimic the therapeutic activity of a CPY peptide, comprising the steps of: (a) conducting a biological assay on a test compound to determine the therapeutic activity; and (b) comparing the results obtained from the biological assay of the test compound to the results obtained from the biological assay of a CPY peptide of claim 1.

14. An isolated nucleic acid which includes a consensus CPY signal sequence set forth in SEQ ID NO:13.

15. A mature CPY peptide derived from the precursor encoded by the nucleic acid sequene of claim 14.
